(19) United States
(12) Patent Application Publication
(10) Pub. No.: US 2015/0051840 A1
(43) Pub. Date: Feb. 19, 2015

(54) Identification Of Microorganisms By Spectrometry And Structured Classification

(71) Applicant: bioMerieux, Marcy L'Etoile (FR)

(72) Inventors: Kevin Vervier, Annoisin-Chatelans (FR); Pierre Mahe, Lans en Vercors (FR); Jean-Baptiste Veyrieras, Lyon (FR)

(21) Appl. No.: 14/387,777

(22) PCT Filed: Apr. 2, 2013
(86) PCT No.: PCT/EP2013/056889

§ 371 Date: Sep. 24, 2014

(30) Foreign Application Priority Data
Apr. 4, 2012 (EP) 12305402.5

(51) Int. Cl. H01J 49/16; G01N 33/50; G06F 19/24

(52) U.S. Cl. CPC H01J 49/164; G06F 19/24; G01N 33/50
USPC 702/19

(57) ABSTRACT

A method of identifying by spectrometry of unknown microorganisms from among a set of reference species, including a first step of supervised learning of a classification model of the reference species, a second step of predicting an unknown microorganism to be identified, including acquiring a spectrum of the unknown microorganism; and applying a prediction model according to said spectrum and to the classification model to infer at least one type of microorganism to which the unknown microorganism belong. The classification model is calculated by a structured multi-class SVM algorithm applied to the nodes of a tree-like hierarchical representation of the reference species in terms of evolution and/or of clinical phenotype and having margin constraints including so-called “loss” functions quantifying a proximity between the tree nodes.

### FIELD OF THE INVENTION

The invention relates to the identification of microorganisms, and particularly bacteria, by means of spectrometry.

The invention can in particular apply in the identification of microorganisms by means of mass spectrometry, for example of MALDI-TOF type (“Matrix-assisted laser desorption ionization time of flight”), of vibrational spectrometry, and of autofluorescence spectroscopy.

### BACKGROUND OF THE INVENTION

It is known to use spectrometry or spectroscopy to identify microorganisms, and more particularly bacteria. For this purpose, a sample of an unknown microorganism is prepared, after which a mass, vibrational, or fluorescence spectrum of the sample is acquired and pre-processed, particularly to eliminate the baseline and to eliminate the noise. The peaks of the pre-processed spectrum are then “compared” by means of classification tools with data from a knowledge base built from a set of reference spectra, each associated with an identified microorganism.

More particularly, the identification of microorganisms by classification conventionally comprises:
a first step of determining, by means of a supervised learning, a classification model according to so-called “training” spectra of microorganisms having their species previously known, the classification model defining a set of rules distinguishing these different species among the training spectra;
a second step of identifying a specific unknown microorganism by:
   acquiring a spectrum thereof; and
   applying to the acquired spectrum a prediction model built from the classification model to determine at least one species to which the unknown microorganism belongs.

Typically, a spectrometry identification device comprises a spectrometer and a data processing unit receiving the measured spectra and implementing the second above-mentioned step. The first step is implemented by the manufacturer of the device who determines the classification model and the prediction model and integrates it in the machine before its use by a customer.

Algorithms of support vector machine or SVM type are conventional supervised learning tools, particularly adapted to the learning of high-dimension classification models aiming at classifying a large number of species.

However, even though SVMs are particularly adapted to high dimension, the determining of a classification model by such algorithms is very complex.

First, conventionally-used SVM algorithms belong to so-called “flat” algorithms which consider the species to be classified equivalently and, as a corollary, also consider classification errors as equivalent. Thus, from an algorithmic viewpoint, a classification error between two close bacteria has the same value as a classification error between a bacteria and a fungus. It is then up to the user, based on his knowledge of the microorganisms used to generate the training spectra, on the structure of the actual spectra, and based on his algorithmic knowledge, to modify the “flat” SVM algorithm used to minimize the severity of the classification errors thereof. Setting aside the difficultly of modifying a complex algorithm, such a modification is highly dependent on the user himself.

Then, even though there would exist some ten or several tens of different training spectra for each microorganism species to build the classification model, this number still remains very low. Not only may the variety of the training spectra be very small as compared with the total variety of the species, but also, a limited number of instances results in mechanically exacerbating the specificity of each spectrum. Thereby, the obtained classification model may be inaccurate for certain species and making the subsequent step of prediction of an unknown microorganism very difficult. Here again, it is up to the user to interpret the results given by the identification to know its degree of relevance and thus, in the end, to deduce an exploitable result therefrom.

### SUMMARY OF THE INVENTION

The present invention aims at providing a method of identifying microorganisms by spectrometry or spectroscopy based on a classification model obtained by an SVM-type supervised learning method which minimizes the severity of identification errors, thus enabling to substantially more reliably identify unknown microorganisms.

For this purpose, an object of the invention is a method of identifying by spectrometry unknown microorganisms from among a set of reference species, comprising:
a first phase of supervised learning of a reference species classification model, comprising:
   for each species, acquiring a set of training spectra of identified microorganisms belonging to said species;
   transforming each acquired training spectrum into a set of training data according to a predetermined format for their use by an algorithm of multi-class support vector machine type; and
   determining the classification model of the reference species as a function of the sets of training data by means of said algorithm of multi-class support vector machine type,
a second step of predicting an unknown microorganism to be identified, comprising:
   acquiring a spectrum of the unknown microorganism; and
   acquiring a spectrum of the unknown microorganism;

According to the invention:
the transforming of each acquired training spectrum comprises:
   transforming the spectrum into a data vector representative of a structure of the training spectrum;
   generating the set of data according to the predetermined format by calculating the tensor product of the data vector by a predetermined vector bijectively representing the position of the reference species of the microorganism in a tree-like hierarchical representation of the reference species in terms of evolution and/or of clinical phenotype;
and the classification model is a classification model with classes corresponding to nodes of the tree of the hierarchical representation, the algorithm of multi-class support vector machine type comprising determining parameters of the classification model by solving a single problem of optimization of a criterion expressed according to the parameters of the classification model under margin constraints comprising so-called “loss functions” quantifying a proximity between the tree nodes.

In other words, the invention specifically introduces a priori information which has not been considered up to now in supervised learning algorithms used in the building of classification models for the identification of microorganisms, that is, a hierarchical tree-like representation of the microorganism species in terms of evolution and/or of clinical phenotype. Such a hierarchical representation is for example a taxonomic tree having its structure essentially guided by the evolution of species, and accordingly which intrinsically contains a notion of similarity or of proximity between species.

The SVM algorithm thus no longer is a “flat” algorithm, the species being no longer interchangeable. As a corollary, classification errors are thus no longer considered identical by the algorithm. By establishing a link between the species to be classified, the method according to the invention thus explicitly and/or implicitly takes into account the fact that they have information in common, and thus also non-common information, which accordingly helps distinguishing species, and thus minimizing classification errors as well as the impact of the small number of training spectra per species.

Such a priori information is introduced into the algorithm by means of a structuring of the data and of the variables due to the tensor product. Thus, the structure of the data and of the variables of the algorithm associated with two species is all the more similar as these species are close in terms of evolution and/or of clinical phenotype. Since SVM algorithms are algorithms aiming at optimizing a cost function under constraints, the optimization thus necessarily takes into account similarities and differences between the structures associated with the species.

In a way, it may be set forth that the proximity between species is “qualitatively” taken into account by the structuring of the data and variables. According to the invention, the proximity between species is also “quantitatively” taken into account by a specific selection of the loss functions involved in the definition of the constraints of the SVM algorithm. Such a “quantitative” proximity of the species is for example determined according to a “distance” defined on the trees of the reference species or may be determined totally independently therefrom, for example, according to specific needs of the user. This thus results in a minimizing of classification errors as well as a gain in robustness of the identification with respect to the paucity of the training spectra.

Finally, the classification model now relates to the classification of the nodes of the tree of the hierarchical representation, including roots and leaves, and no longer only to species. Particularly, if during a prediction implemented on the spectrum of an unknown microorganism, it is difficult to determine the species to which the microorganism belongs with a minimum degree of certainty, the prediction is capable of identifying to which larger group (genus, family, order . . . ) of microorganisms the unknown microorganism belongs. Such precious information may for example be used to implement other types of microbial identifications specific to said identified group.

According to an embodiment, loss functions associated with pairs of nodes are equal to distances separating the nodes in the tree of the hierarchical representation. Thereby, the algorithm is optimized for said tree, and the loss functions do not depend on the user's know-how and knowledge.

According to an embodiment, loss functions associated with pairs of nodes are respectively greater than distances separating the nodes in the tree of the hierarchical representation. Thus, another type of a priori information may be introduced in the building of the classification model. Particularly, the algorithmic separability of the species may be forced by selecting loss functions having a value greater than the distance in the tree.

According to an embodiment, the loss functions are calculated:
by setting the loss functions to initial values;
by implementing at least one iteration of a process comprising:
   executing an algorithm of multi-class support vector machine type to calculate a classification model according to current values of the loss functions;
   applying a prediction model according to the calculated classification model and to a set of calibration spectra of identified microorganisms belonging to the reference species, different from the set of training spectra;
   calculating a classification performance criterion for each species according to results returned by said application of the prediction model to the set of calibration spectra; and
   calculating new current values of the loss functions by modifying the current values of the loss functions according to the calculated performance criteria.

The loss functions particularly enable to set the separability of the species regarding the training spectra and/or the used SVM algorithm. It is in particular possible to detect species with a low separability and to implement an algorithm which modifies the loss functions to increase this separability.

In a first variation:
the calculation of the performance criterion comprises calculating a confusion matrix as a function of the results returned by said application of the prediction model;
and the new current values of the loss functions are calculated as a function of the confusion matrix.

Thereby, the impact of having introduced the taxonomy and/or clinical phenotype information contained in the tree of the hierarchical representation is assessed and the remaining errors or classification defects are minimized by selecting loss functions as a function thereof.

According to a second variation:
the calculation of the performance criterion comprises calculating a confusion matrix as a function of the results returned by said application of the prediction model;
and the new current values of the loss functions respectively correspond to the components of a combination of a first loss matrix listing distances separating the reference species in the tree of the hierarchical representation and of a second matrix calculated as a function of the confusion matrix.

Just as in the first variation, the remaining error and classification defects are corrected while keeping in the loss functions quantitative information relative to the distances between species in the tree.

Particularly, the current values of the loss functions are calculated according to relation:

Δ(yᵢ, k)=α×Ω(yᵢ, k)+(1−α)×Δ_{confusion}(yᵢ, k)

where Δ(yᵢ, k) are said current values of the loss functions for node pairs (yᵢ, k) of the tree, Ω(yᵢ, k) and Δ_{confusion}(yᵢ, k) respectively are the first and second matrixes, and α is a scalar number between 0 and 1. More particularly, a is in the range from 0.25 to 0.75, particularly from 0.25 to 0.5.

Such a convex combination provides both a high accuracy of the identification and a minimization of the severity of identification errors.

More particularly, the initial values of the loss functions are set to zero for pairs of different nodes and equal to 1 otherwise.

According to an embodiment, a distance Ω separating two nodes n₁, n₂ in the tree of the hierarchical representation is determined according to relation:

Ω(n₁, n₂)=depth (n₁)+depth (n₂)−2×depth (LCA (n₁, n₂))

where depth (n₁) and depth (n₂) respectively are the depth of nodes n₁, n₂ , and depth (LCA (n₁, n₂)) is the depth of the closest common ancestor LCA (n₁, n₂) of nodes n₁, n₂ in said tree. Distance Ω thus defined is the minimum distance capable of being defined in a tree.

According to an embodiment, the prediction model is a prediction model for the tree nodes to which the unknown microorganism to be identified belongs. It is thus possible to predict nodes which are ancestors to the leaves corresponding to the species.

According to an embodiment, the optimization problem is formulated according to relations:

$${min}_{W , {ξ}_{i}} \frac{1}{2} {‖ W ‖}^{2} + C {\sum }_{i = 1}^{N} {ξ}_{i}$$

under constraints:

ξᵢ≧0, ∀i ∈ [1, N]

[symbol]

W, Ψ(xᵢ, yᵢ) [symbol] ≧ [symbol] W, Ψ(xᵢ, k) [symbol] +∫(Δ(yᵢ, k), ξᵢ), ∀i ∈ [1, N], ∀k ∈ Y \yᵢ

in which expressions:
N is the number of training spectra;
K is the number of reference species;

T is the number of nodes in the tree of the hierarchical representation and Y=[1, T] is a set of integers used as reference numerals for the nodes of the tree of the hierarchical representation;
W ∈ [symbol] ^{p×T} is the concatenation (w₁w_{w} . . . w_{T})^{T} of weight vectors w₁, w₂, . . . , w_{T} ∈ [symbol] ^{p} respectively associated with the nodes of said tree, p being the cardinality of the vectors representative of the structure of the training spectra;
C is a scalar having a predetermined setting;
∀i ∈ [1, N], ξᵢ is a scalar;
X={xᵢ}, i ∈ [1, N] is a set of vectors xᵢ ∈ [symbol] ₚ representative of the training spectra;
∀i ∈ [1, N], yᵢ is the reference numeral of the node in the tree of the hierarchical representation corresponding to the reference species of training vector xᵢ ;
Ψ(x,k)=x [symbol] Λ(k), where:
   x ∈ [symbol] ^{p} is a vector representative of a training spectrum;
   Λ(k) ∈ [symbol] ^{T} is a predetermined vector bijectively representing the position of reference node k ∈ Y in the tree of the hierarchical representation; and
   [symbol] : [symbol] ^{p}× [symbol] ^{p×T} is the tensor product of space [symbol] ^{P} and space [symbol] ^{T};
[symbol] W, Ψ [symbol] is the scalar product over space [symbol] ^{p×T};
Δ(yᵢ,k) is the loss function associated with the pair of nodes bearing respective references yᵢ and k in the tree of the hierarchical representation;
f (Δ(yᵢ, k),ξᵢ) is a predetermined function of scalar εᵢ and of loss function Δ(yᵢ, k); and
symbol “\” designates exclusion.

In a first variation, function f (Δ(yᵢ, k),εᵢ) is defined according to relation f(Δ(yᵢ,k),εᵢ)=Δ(yᵢ, k)−εᵢ. In a second variation, function f (Δ(yᵢ, k),εᵢ) is defined according to relation

$$f ( Δ ( {y}_{i} , k ) , {ξ}_{i} ) = 1 - \frac{{ξ}_{i}}{Δ ( {y}_{i} , k )} .$$

Particularly, the prediction step comprises:
transforming the spectrum of the unknown microorganism to be identified into a vector xₘ, according to the predetermined format of the algorithm of multi-class support vector machine type;
applying a prediction model according to relations:

T_{indent}=arg maxₖ (s(xₘ, k)) k ∈ [1, T]

where T_{indent} is the reference of the node of the hierarchical representation identified for the unknown microorganism,

s(xₘ, k)= [symbol] W, Ψ(xₘ, k) [symbol] and Ψ (xₘ, k)=xₘ [symbol] Λ(k).

The invention also aims at a method of identifying a microorganism by mass spectrometry, comprising:
a spectrometer capable of generating mass spectra of microorganisms to be identified;
a calculation unit capable of identifying the microorganisms associated with the spectra generated by the spectrometer by implementing a prediction step of the above-mentioned type.

### BRIEF DESCRIPTION OF THE DRAWINGS

The present invention will be better understood on reading of the following description provided as an example only in relation with the accompanying drawings, where the same reference numerals designate the same or similar elements, among which:

FIG. 1 is a flowchart of an identification method according to the invention;

FIG. 2 is an example of a hybrid taxonomy tree for example mixing phenotype and evolution information;

FIG. 3 is an example of a tree of a hierarchical representation used according to the invention;

FIG. 4 is an example of generation of a vector corresponding to the position of a node in a tree;

FIG. 5 is a flowchart of a loss function calculation method according to the invention;

FIG. 6 is a plot illustrating accuracies per species of different identification algorithms;

FIG. 7 is a plot illustrating taxonomic costs of prediction errors of these different algorithms;

FIG. 8 is a plot illustrating accuracies per species of an algorithm using loss functions equal to different convex combinations of a distance in the tree of the hierarchical representation and of a confusion loss function; and

FIG. 9 is a plot of the taxonomic costs of prediction errors for the different convex combinations.

### DETAILED DESCRIPTION OF THE INVENTION

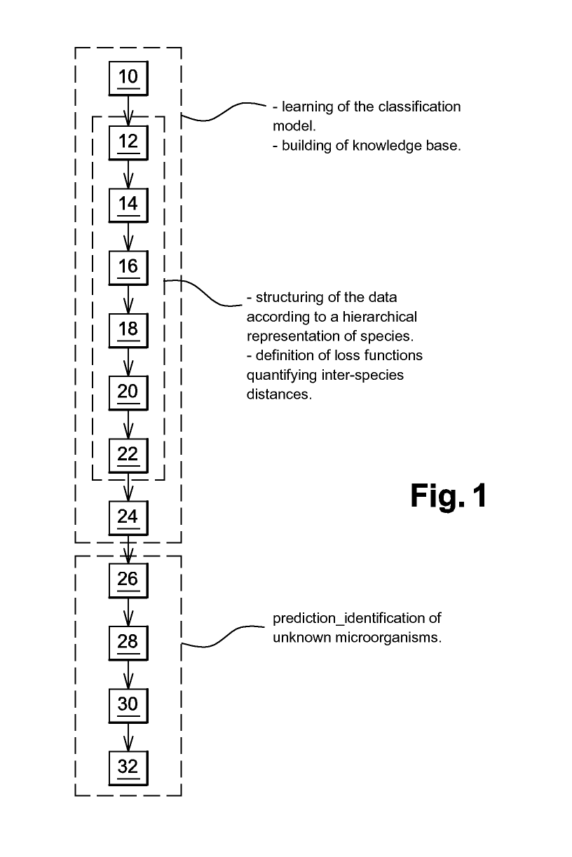

A method according to the invention applied to MALDI-TOF spectrometry will now be described in relation with the flowchart of FIG. 1.

The method starts with a step 10 of acquiring a set of training mass spectra of a new microorganism species to be integrated in a knowledge base, for example, by means of a MALDI-TOF (“Matrix-assisted laser desorption/ionization time of flight”) mass spectrometry. MALDI-TOF mass spectrometry is well known per se and will not be described in further detail hereafter. Reference may for example be made to Jackson O. Lay's document, “Maldi-tof spectrometry of bacteria”, Mass Spectrometry Reviews, 2001, 20, 172-194. The acquired spectra are then preprocessed, particularly to denoise them and remove their baseline, as known per se.

The peaks present in the acquired spectrum are then identified at step 12, for example, by means of a peak detection algorithm based on the detection of local maximum values. A list of peaks for each acquired spectrum, comprising the location and the intensity of the spectrum peaks, is thus generated.

Advantageously, the peaks are identified in the predetermined range of Thomson [mₘᵢₙ;mₘₐₓ], preferably Thomson's range [mₘᵢₙ;mₘₐₓ]=[3,000;17,000]. Indeed, it has been observed that the information sufficient to identify the microorganisms is contained in this range of mass-to-charge ratios, and that it is thus not needed to take a wider range into account.

The method carries on, at step 14, by a quantization or “binning” step. To achieve this, range [mₘᵢₙ;mₘₐₓ] is divided into intervals of predetermined widths, for example, constant, and for each interval comprising a plurality of peaks, a single peak is kept, advantageously the peak having the highest intensity. A vector is thus generated for each measured spectrum. Each component of the vector corresponds to a quantization interval and has, as a value, the intensity of the peak kept for this interval, value “0” meaning that no peak has been detected in the interval.

As a variation, the vectors are “binarized” by setting the value of a component of the vector to “1” when a peak is present in the corresponding interval, and to “0” when no peak is present in this interval. This results in increasing the robustness of the subsequently-performed classification algorithm calibration. The inventors have indeed noted that the information relevant, particularly, to identify a bacterium is essentially contained in the absence and/or the presence of peaks, and that the intensity information is less relevant. It can further be observed that the intensity is highly variable from one spectrum to the other and/or from one spectrometer to the other. Due to this variability, it is difficult to take into account raw intensity values in the classification tools.

In parallel, the training spectrum peak vectors, called “training vectors” hereafter, are stored in the knowledge base. The knowledge base thus lists K microorganism species, called “reference species”, and one set X={xᵢ}_{i∈ [1,N]} of N training spectra xᵢ ∈ [symbol] ^{P}, i ∈ [1, N], where p is the number of peaks retained for the mass spectra.

At the same time, or consecutively, the listed species K are classified, at 16, according to a tree-like hierarchical representation of reference species in terms of evolution and/or of clinical phenotype.

In a first variation, the hierarchical representation is a taxonomic representation of living beings applied to the listed reference species. As known per se, the taxonomy of living organisms is a hierarchical classification of living beings which classifies each living organism according to the following order, from the least specific to the most specific: domain, kingdom, phylum, class, order, family, genus, species. The taxonomy used is for example that determined by the “National Center for Biotechnology Information” (NCBI). The taxonomy of living organisms thus implicitly comprises evolutionary data, close microorganisms at an evolutionary level comprising more components in common than microorganisms that are more remote in terms of evolution. Thereby, the evolutionary “proximity” has an impact on the “proximity” of spectra.

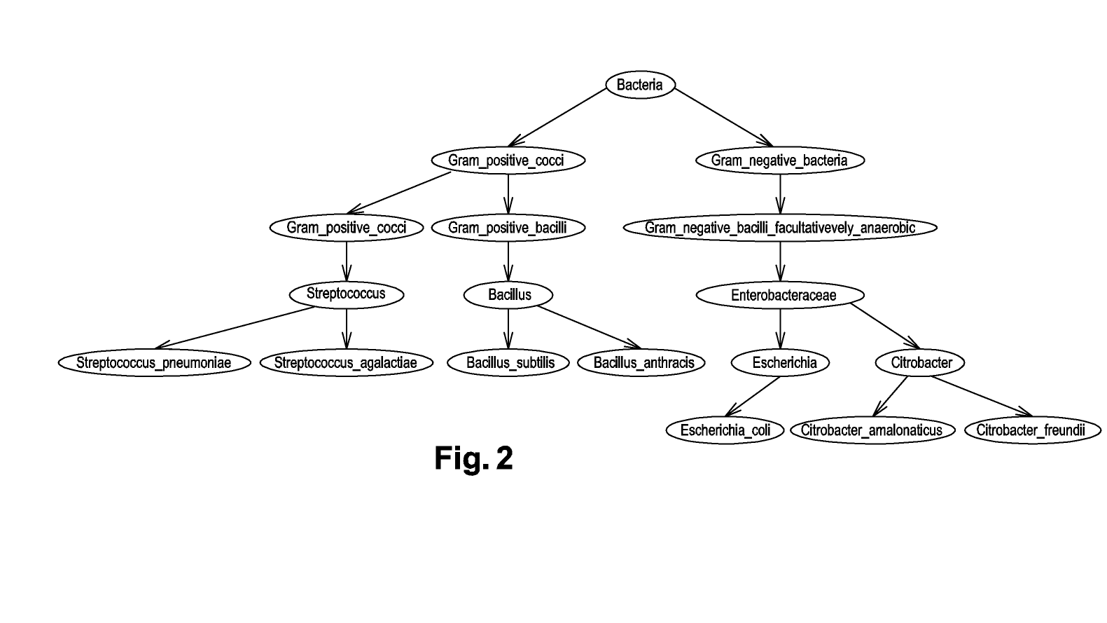

In a second variation, the hierarchical representation is a “hybrid” taxonomic representation obtained by taking into account phylogenic characteristics, for example, species evolution characteristics, and phenotype characteristics, such as for example the GRAM +/− of the bacteria, which is based on the thicknesspermeability of their membranes, their aerobic or anaerobic characteristic. Such a representation is for example illustrated in FIG. 2 for bacteria.

Generally, the tree of the hierarchical representation is a graphical representation connecting end nodes, or “leaves”, corresponding to the species to a “root” node by a single path formed of intermediate nodes.

At a next step 18, the tree nodes, or “taxons”, are numbered with integers k ∈ Y=[1, T], where T is the number of nodes in the tree, including leaves and roots, and the tree is transformed into a set Λ={Λ(k)}_{k ∈ [1, T]} of binary vectors Λ(k) ∈ [symbol] ^{T}.

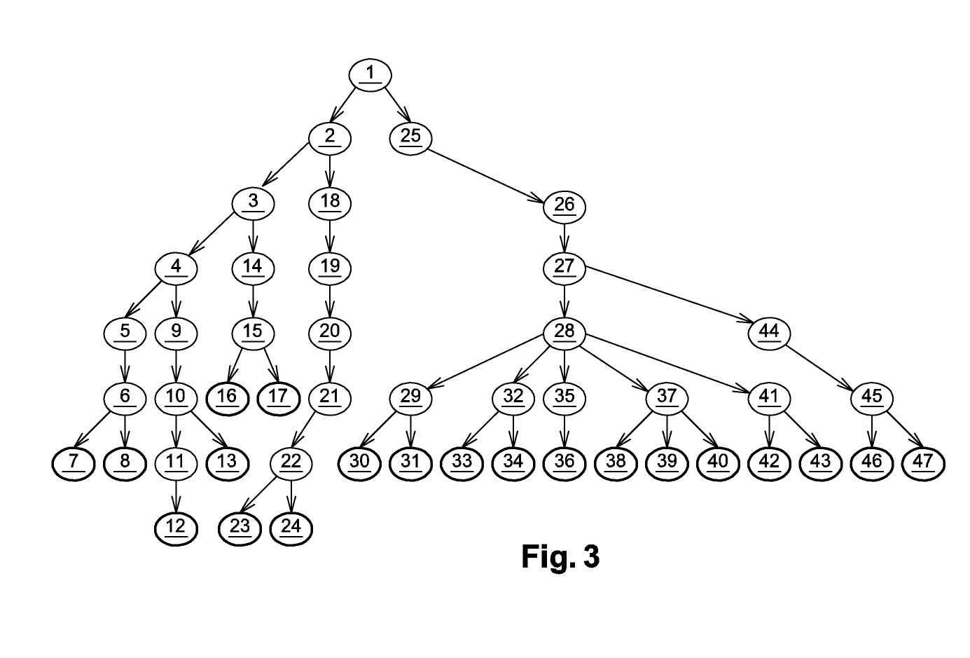

More particularly, nodes T of the tree are respectively numbered from 1 to T, for example, in accordance with the different paths from the root to the leaves, as illustrated in the tree of FIG. 3 which lists 47 nodes, among which 20 species. The components of vectors Λ(k) then correspond to the nodes thus numbered, the first component of vectors Λ(k) corresponding to the node bearing number “1”, the second component corresponding to the node bearing number “2”, and so on. The components of a vector Λ(k) corresponding to the nodes in the path from node k to the root of the tree, including node k and the root, are set to be equal to one, and the other components of vector Λ(k) are set to be equal to zero. FIG. 4 illustrates the generator of vectors Λ(k) for simplified tree of 5 nodes. Vector Λ(k) thus bijectively or uniquely represents the position of node k in the tree of the hierarchical representation, and the structure of vector Λ(k) represents the ascendancy links of node k. In other words, set Λ={Λ(k)}_{k ∈ [1, T]}is a vectorial representation of all the paths between the root and the nodes of the tree of the hierarchical representation.

Other vectorial representations of the tree keeping these links are of course possible.

To better understand the following, the following notations are introduced. Each training vector xᵢ corresponds to a specific reference species labeled with an integer yᵢ ∈ [1, T], that is, the number of the corresponding leaf in the tree of the hierarchical representation. For example, the 10^{th} training vector x₁₀ corresponds to the species represented by leaf number “24” of the tree of FIG. 3, in which case y₁₀=24. Notation yᵢ thus refers to the number, or “label” of the species of the spectrum in set [1, T], the cardinality of the set E={yᵢ} of reference numerals yᵢ being of course equal to number K of reference species. Thus, referring, for example, to FIG. 3, E={7,8,12,13,16,17,23,24,30,31,33,34,36,38,39,40,42,43,46,47}. When an integer from Y=[1, T], for example, integer “K”, is directly used in the following relations, this integer refers to the node bearing number “K” in the tree, independently from training vectors xᵢ.

At a next step 20, new “structured training” vectors Ψ (xᵢ, k) ∈ [symbol] ^{p×T} are generated according to relations:

Ψ(xᵢ,k)=xᵢ [symbol] Λ(k) ∀i ∈ [1, N], ∀k ∈ [1, T] (1)

where [symbol] : [symbol] ^{p}× [symbol] ^{T}→ [symbol] ^{p×T} is the tensor product between space [symbol] ^{p} and space [symbol] ^{T}. A vector Ψ(xᵢ, k) thus is a vector which comprises a concatenation of T blocks of dimension p where the blocks corresponding to the components equal to one unit of vector Λ(k) are equal to vector xᵢ and the other blocks are equal to zero vector 0_{P} of [symbol] ^{P}. Referring again to the example of FIG. 4, vector Λ(5) corresponding to node number
“5” is equal to

$$( \begin{matrix}1 \\ 0 \\ 1 \\ 0 \\ 1 \\ 0\end{matrix} )$$

and vector Ψ(xᵢ,5) is equal to

$$( \begin{matrix}{x}_{i} \\ {0}_{p} \\ {x}_{i} \\ {0}_{p} \\ {x}_{i} \\ {0}_{p}\end{matrix} )$$

It can thus be observed that the closer nodes are to one another in the tree of the hierarchical representation, the more their structured vectors share common non-zero blocks. Conversely, the more nodes are remote, the less their structured vectors share non-zero blocks in common, such observations thus in particular applying to leaves representing reference species.

At a next step 22, loss functions of a structured multi-class SVM type algorithm applied to all the nodes of the tree of the hierarchical representation are calculated.

More particularly, a multi-class SVM algorithm structured in accordance with the hierarchical representation according to the invention is defined according to relations:

$${min}_{W , {ξ}_{i}} \frac{1}{2} {‖ W ‖}^{2} + C {\sum }_{i = 1}^{N} {ξ}_{i} \qquad ( 2 )$$

under constraints:

εᵢ≧0, ∀i ∈ [1, N] (3)

[symbol]

W, Ψ (xᵢ, yᵢ) [symbol] ≧ [symbol] W, Ψ (xᵢ, k) [symbol] +f (Δ(yᵢ, k), ξᵢ), ∀i ∈ [1, N], ∀k ∈ Y\yᵢ (4)

in which expressions:
W ∈ [symbol] ^{p×T} is the concatenation (w₁w₂ . . . w_{T})^{T} of weight vectors w₁,w₂ . . . , w_{T} ∈ [symbol] ^{p} respectively associated with nodes yᵢ of the tree;
C is a scalar having a predetermined setting;
C is a scalar having a predetermined setting;
[symbol] W, Ψ [symbol] is the scalar product, here over space [symbol] ^{p×T};
Δ(yᵢ,k) is a loss function defined for the pair formed by the species bearing reference yᵢ and the node bearing reference k;
f(Δ(yᵢ,k),ξᵢ) is a predetermined function of scalar ξᵢ and of loss function Δ(yᵢ, k); and
symbol “\” designating exclusion, expression “∀k ∈ Y\yᵢ” thus meaning “all the nodes of set Y except reference node yᵢ”.

As can be observed, the proximity between species, such as coded by the hierarchical representation, and such as introduced into the structure of the structured training vector, is taken into account via the constraints. Particularly, the closer species are to one another in the tree, the more their data are coupled. The reference species are thus no longer considered as interchangeable by the algorithm according to the invention, conversely to conventional multi-class SVM algorithms, which consider no hierarchy between species and consider said species as being interchangeable.

Further, the structured multi-class SVM algorithm according to the invention quantitatively takes into account the proximity between reference species by means of loss functions Δ(yᵢ, k).

According to a first variation, function f is defined according to relation:

f (Δ(yᵢ, k),ξᵢ)=Δ(yᵢ, k)−ξᵢ (5)

According to a second variation, function f is defined according to relation:

$$f ( Δ ( {y}_{i} , k ) , {ξ}_{i} ) = 1 - \frac{{ξ}_{i}}{Δ ( {y}_{i} , k )} \qquad ( 6 )$$

In an advantageous embodiment, loss functions Δ(yᵢ, k) are equal to a distance Ω(yᵢ, k) defined in the tree of the hierarchical representation according to relation:

Δ(yᵢ, k)=Ω(yᵢ, k)=depth(yᵢ)+depth(k)−2×depth(LCA(yᵢ, k)) (7)

where depth(yᵢ) and depth(k) respectively are the depth of nodes yᵢ and k in said tree, and depth(LCA(yᵢ, k)) is the depth of the ascending node, or closest common “ancestor” node LCA(yᵢ,k) of nodes yᵢ , k in said tree. The depth of a node is for example defined as being the number of nodes which separate it from the root node.

As a variation, loss functions Δ(yᵢ,k) are of a nature different from that of the hierarchical representation. These functions are for example defined by the user according to another hierarchical representation, to his know-how and/or to algorithmic results, as will be explained in further detail hereafter.

Once the loss functions have been calculated, the method according to the invention carries on with the implementation, at 24, of the multi-class SVM algorithm such as defined in relations (2), (3), (4), (5) or (2), (3), (4), (6).

The result produced by the algorithm thus is vector W which is the classification model of the tree nodes, deduced from the combination of the information contained in training vectors xᵢ , from the positioning of their associated reference species in the tree, from the information as to the proximity between species contained in the hierarchical representation, and from the information as to the distance between species contained in the loss functions. More particularly, each weight vector wᵢ, l ∈ [1, T] represents the normal vector of a hyperplane of [symbol] ^{p} forming a border between the instances of node “l” of the tree and the instances of the other nodes k ∈ [1, T]\1 of the tree.

Training steps 12 to 24 of the classification model are implemented once in a first computer system. Classification model W=(w₁w₂ . . . w_{T})^{T} and vectors Λ(k) are then stored in a microorganism identification system comprising a MALDI-TOF-type spectrometer and a computer processing unit connected to the spectrometer. The processing unit receives the mass spectra acquired by the spectrometer and implements the production rules determining, based on model W and on vectors Λ(k), to which nodes of the tree of the hierarchical representation the mass spectra acquired by the mass spectrometer are associated.

As a variation, the prediction is performed on a distant server accessible by a user, for example, by means of a personal computer connected to the Internet to which the server is also connected. The user loads non-processed mass spectra obtained by a MALDI-TOF type mass spectrometer onto the server, which then implements the prediction algorithm and returns the results of the algorithm to the user's computer.

More particularly, for the identification of an unknown microorganism, the method comprises a step 26 of acquiring one or a plurality of mass spectra thereof, a step 28 of preprocessing the acquired spectra, as well as a step 30 of detecting peaks of the spectra and of determining a peak vector xₘ ∈ [symbol] ^{p}, such as for example previously described in relation with steps 10 to 14.

At a next step 32, a structured vector is calculated for each node in the tree of the hierarchical representation, k ∈ Y=[1, T], according to relation:

Ψ(xₘ, k)=xₘ [symbol] Λ(k) (8)

after which a score associated with node k is calculated according to relation:

s(xₘ, k)= [symbol] W, Ψ(xₘ, k) [symbol] (9)

The identified node of tree T_{indent} ∈ [1, T] of the unknown microorganism then for example is that which corresponds to the highest score:

T_{indent}=arg maxₖ (s(xₘ, k)) k ∈ [1, T] (10)

Other prediction models are of course possible.

Apart from the score associated with identified taxon T_{indent}, the scores of the ancestor nodes and of the daughter nodes, if they exist, of taxon T_{indent} are also calculated by the prediction algorithm. Thus, for example, if the score of taxon T_{indent} is considered as low by the user, the latter has scores associated with the ancestor nodes, and thus additional more reliable information.

A specific embodiment of the invention where loss functions Δ(yᵢ,k) are calculated according to a minimum distance defined in the tree of the hierarchical representation has just been described.

Other alternative calculations of loss functions Δ(yᵢ, k) will now be described.

In a first variation, the loss functions defined at relation (7) are modified according to a priori information enabling to obtain a more robust classification model and/or to ease the resolution of the optimization problem defined by relations (2), (3), and (4). For example, the loss function Δ(yᵢ, k) of a pair of nodes (yᵢ,k) may be selected to be low, in particular smaller than distance Ω(yᵢ, k) , which means that identification errors are tolerated between these two nodes. Releasing constraints on one or a plurality of pairs of species mechanically amounts to increasing constraints on the other pairs of species, the algorithm being then set to more strongly differentiate the other pairs. Similarly, loss function Δ(yᵢ,k) of a pair of nodes (yᵢ,k) may be selected to be very high, particularly greater than distance Ω(yᵢ, k), to force the algorithm to differentiate nodes (yᵢ, k), and thus to minimize identification errors therebetween. In particular, it is possible to release or to reinforce constraints bearing on pairs of reference species by means of their respective loss functions.

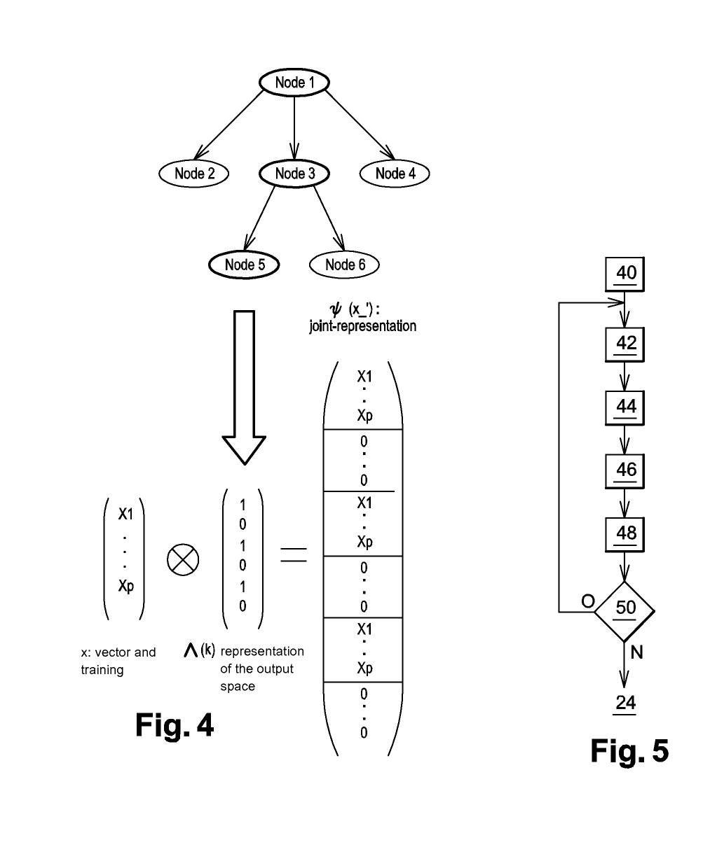

In a second variation, illustrated in the flowchart of FIG. 5, the calculation of loss functions Δ(yᵢ, k) is performed automatically according to the estimated performance of the SVM algorithm implemented to calculate classification model W.

The method of calculating loss functions Δ(yᵢ, k) starts with the selection, at 40, of initial values for them. For example, Δ(yᵢ, k)=0 when yᵢ=k , and Δ(yᵢ, k)=1 when yᵢ≠k, functions f thus being reduced to f (Δ(yᵢ, k),ξᵢ)=1−ξᵢ. Other initial values are of course possible for the loss functions, functions f(ξᵢ)=1−ξᵢ appearing in the constraints of the above-discussed algorithms being then replaced with functions f (Δ(yᵢ,k),ξᵢ) of relation (5) or (6) with the initial values of the loss functions.

The calculation method carries on with the estimation of the performance of the SVM algorithm for the selected loss functions Δ(yᵢ, k). Such an estimation comprises:
executing, at 42, a multi-class SVM algorithm according to the values of the loss functions to calculate a classification model;
applying, at 44, a prediction model based on the calculated classification model, the prediction model being applied to a set {\tilde{x}ᵢ} of calibration vectors \tilde{x}ᵢ ∈ [symbol] ^{p} of the knowledge base. Calibration vectors \tilde{x}ᵢ are generated similarly to training vectors xᵢ from spectra associated with the reference species, each vector \tilde{x}ᵢ being associated with reference \tilde{y}ᵢ of the corresponding reference species; and
determining, at 46, a confusion matrix according to the results of the prediction.

Calibration vectors \tilde{x}ᵢ are for example acquired at the same time as training vectors xᵢ . Particularly, for each reference species, the spectra associated therewith are distributed into a training set and a calibration set from which the training vectors and the calibration vectors are respectively generated.

The loss function calculation method carries on, at 48, with the modification of the values of the loss functions according to the calculated confusion matrix. The obtained loss functions are then used by the SVM algorithm for calculating final classification model W , or a test is carried out at 50 to know whether new values of the loss functions are calculated by implementing steps 42, 44, 46, 48 according to values of the loss functions modified at step 48.

In a first example of the loss function calculation method, step 42 corresponding to the execution of an SVM algorithm is a one-versus-all type algorithm. This algorithm is not hierarchical and only considers the reference species, referred to with integers k ∈ [1, K] , and solves a problem of optimization for each of reference species k according to relations:

$${min}_{{w}_{k} , {ξ}_{i}} \frac{1}{2} {‖ {w}_{k} ‖}^{2} + C {\sum }_{i = 1}^{N} {ξ}_{i} \qquad ( 11 )$$

under constraints:

ξᵢ≧0, ∀i ∈ [1, N] (12)

qᵢ( [symbol] wₖ, xᵢ [symbol] +bₖ)≧1−ξᵢ ∀i ∈ [1, N] (13)

in which expressions:
wₖ ∈ [symbol] ^{p} is a weight vector and bₖ ∈ [symbol] is a scalar;
qᵢ ∈ {−1,1} with qᵢ=1 if i=k, and qᵢ=−1 if i≠k.

The prediction model is provided by the following relation and applied, at step 44, to each of calibration vectors \tilde{x}ᵢ:

G(\tilde{x}ᵢ)=arg maxₖ [symbol] wₖ, \tilde{x}ᵢ [symbol] +bₖ k ∈ [1, K] (14)

An inter-species confusion matrix C_{species} ∈ [symbol] ^{K}× [symbol] ^{K} is then calculated, at step 46, according to relation:

C_{species}(i,k)=FP(i,k) ∀i, k ∈ [1, K] (15)

where FP(i,k) is the number of calibration vectors of species i predicted by the prediction model as belonging to species k.

Still at 46, a normalized inter-species confusion matrix \tilde{C}_{species} ∈ [symbol] ^{K}× [symbol] ^{K} is then calculated according to relation:

$${\tilde{C}}_{species} ( i , k ) = \frac{{C}_{species} ( i , k )}{{N}_{i}} \times 100 \qquad ( 16 )$$

where N is the number of calibration vectors for the species bearing reference i.

Finally, step 46 ends with the calculation of a normalized inter-node confusion matrix \tilde{C}ₜₐₓₒ ∈ [symbol] ^{T}× [symbol] ^{T} as a function of normalized confusion matrix \tilde{C}_{species}. For example, a propagation diagram of values \tilde{C}_{species}(i,k) from the leaves to the root is used to calculate values \tilde{C}ₜₐₓₒ(i,k) of pairs (i,k) of different nodes of the reference species. Particularly, for a pair of nodes (i,k) ∈ [1, T]² of the tree of the hierarchical representation for which a component of matrix \tilde{C}ₜₐₓₒ(i^{C},k^{C}) has already been calculated for each pair of nodes (i^{C},k^{C}) of set {i^{C}}×{k^{C}}, where {i^{C}} and {k^{C}} respectively are the sets of “daughter” nodes of nodes i and k, the component of matrix \tilde{C}ₜₐₓₒ(i^{C},k^{C}) for pair (i,k) is set to be equal to the average of components \tilde{C}ₜₐₓₒ(i^{C},k^{C}).

At step 48, the loss function Δ(yᵢ,k) of each pair of nodes (yᵢ,k) is calculated as a function of normalize inter-node confusion matrix \tilde{C}ₜₐₓₒ.

According to a first option of step 48, loss function Δ(yᵢ,k) is calculated according to relation:

$$Δ ( {y}_{i} , k ) = \{ \begin{matrix}0 & if {y}_{i} = k \\ 1 + λ \times {\tilde{C}}_{taxo} ( {y}_{i} , k ) & if {y}_{i} \neq k\end{matrix} \qquad ( 17 )$$

where λ≧0 is a predetermined scalar controlling the contribution of confusion matrix \tilde{C}ₜₐₓₒ in the loss function.

According to a second option of step 48, loss function Δ(yᵢ,k) is calculated according to relation:

$$Δ ( {y}_{i} , k ) = \{ \begin{matrix}0 & if {y}_{i} = k \\ 1 + β \times ⌈ \frac{{\tilde{C}}_{taxo} ( {y}_{i} , k )}{l} ⌉ & if {y}_{i} \neq k\end{matrix} \qquad ( 18 )$$

where ┌•┐ is the rounding to the next highest integer, β≧0 and l>0 are predetermined scalars setting the contribution of confusion matrix \tilde{C}ₜₐₓₒ in the loss function. For example, by setting l=10, confusion matrix \tilde{C}ₜₐₓₒ contributes by β per 10% of confusion between nodes (yᵢ,k).

According to a third option of step 48, a first component Δ_{confusion} (yᵢ, k) of loss function Δ(yᵢ,k) is calculated according to relation (17) or (18), after which loss function Δ(yᵢ,k) is calculated according to relation:

Δ(yᵢ,k)=α×Ψ(yᵢ, k)+(1−α)×Δ_{confusion} (yᵢ,k) (19)

where 0≦α≦1 is a scalar setting a tradeoff between a loss function only determined by means of a confusion matrix and a loss function only determined by means of a distance in the tree of the hierarchical representation.

In a second example of the loss function calculation method, step 42 corresponds to the execution of a multi-class SVM algorithm which solves a single optimization problem for all references species k ∈ [1, K], each training vector xᵢ being associated with its reference species bearing as a reference number an integer yᵢ ∈ [1, K], according to relations

$${min}_{{w}_{k} , {ξ}_{i}} \frac{1}{2} {\sum }_{k = 1}^{K} {‖ {w}_{k} ‖}^{2} + C {\sum }_{i = 1}^{N} {ξ}_{i} \qquad ( 20 )$$

under constraints:

ξᵢ≧0, ∀i ∈ [1, N] (21)

[symbol]

w_{y}_{ᵢ}, xᵢ [symbol] ≧ [symbol] wₖ, xᵢ [symbol] +1−ξᵢ ∀i ∈ [1, N], ∀k ∈ [1, K]\yᵢ (22)

where ∀k ∈ [1, K], wₖ ∈ [symbol] ^{p} is a weight vector associated with species k.

The prediction model is provided by the following relation and applied, at step 44, to each of calibration vectors \tilde{x}ᵢ:

G(\tilde{x}ᵢ)=arg maxₖ [symbol] wₖ, \tilde{x}ᵢ [symbol] k ∈ [1, K] (23)

Steps 46 and 48 of the second example are identical to steps 46 and 48 of the first example.

In a third example of the loss function calculation method, step 42 corresponds to the execution of structured multi-class SVMs based on a hierarchical representation according to relations (2), (3), (4), (5) or (2), (3), (4), (6). At step 44, the prediction model according to the following relation is then applied to each of calibration vectors \tilde{x}ᵢ:

G(\tilde{x}ᵢ)=arg maxₖ [symbol] W, Ψ(\tilde{x}ᵢ, k) [symbol] k ∈ E (29)
where E={yₖ^{species}} is the set of references of the nodes of the tree of the hierarchical representation corresponding to the reference species.

An inter-species confusion matrix C_{species} ∈ [symbol] ^{K}× [symbol] ^{K} is then deduced from the results of the prediction on calibration vectors \tilde{x}ᵢ and the loss function calculation method carries on identically to that of the first example.

Of course, the confusion may be calculated according to prediction results bearing on all the taxons in the tree.

Embodiments where the SVM algorithm implemented to calculate the classification model is a structured multi-class SVM model based on a hierarchical representation, particularly an algorithm according to relations (2), (3), (4), (5) or according to relations (2), (3), (4), (6), have been described.

The principle of loss functions Δ(yᵢ,k) which quantify an a priori proximity between classes envisaged by the algorithm, that is, nodes of the tree of the hierarchical representation in the previously-described embodiments, also apply to multi-class SVM algorithms which are not based on a hierarchical representation. For such algorithms, the considered classes are the reference species represented in the algorithms by integers k ∈ [1, K], and the loss functions are only defined for the pairs of reference species, and thus for couples (yᵢ,k)∈ [1, K]².

Particularly, in another embodiment, the SVM algorithm used to calculate the classification model is the multi-class SVM algorithm according to relations (20), (21), and (22), replacing function f(ξᵢ)=1−ξᵢ of relation (22) with function f (Δ(yᵢ, k),ξᵢ) according to relation (5) or relation (6), that is, according to relations (20), (21), and (22bis):

[symbol]

w_{y}_{ᵢ}, xᵢ [symbol] ≧ [symbol] wₖ, xᵢ [symbol] +f(Δ(yᵢ, k), ξᵢ), ∀i ∈ [1, N], ∀k ∈ [1, K]\yᵢ (22bis)

The prediction model applied to identify the species of an unknown microorganism then is the model according to relation (23).

Experimental results of the method according to the invention will now be described, in the following experimental conditions:
571 spectra of bacteria obtained by a MALDI-TOF-type mass spectrometer;
the bacteria belong to 20 different reference species and correspond to more than 200 different strains; and
the 20 species are hierarchically organized in a taxonomic tree of 47 nodes such as illustrated in FIG. 3;
the training and calibration vectors are generated according to the mass spectra and each list the intensity of 1,300 peaks according to the mass-to-charge ratio. Thus, xᵢ ∈ [symbol] ¹³⁰⁰.

The performance of the method according to the invention is assessed by means of a cross-validation defined as follows:
for each strain, a set of training vectors is defined by removing from the total set of training vectors the vectors corresponding to the strain;
for each set thus obtained, a classification model is calculated based on a SVM-type algorithm such as described hereabove; and
a prediction model associated with the obtained classification model is applied to the vectors corresponding to the strain removed from the set of training vectors.

Further, different indicators are taken into account to assess the performance of the method:
the micro-accuracy, which is the ratio of properly classified spectra;
accuracies per species, an accuracy for a species being the ratio of properly-classified spectra for this species;
the macro-accuracy, which is the average of the accuracies per species. Unlike micro-accuracy, macro-accuracy is less sensitive to the cardinality of the sets of training vectors respectively associated with the reference species;
the “taxonomy” cost of a prediction, which is the length of the shortest path in the tree of the hierarchical representation between the reference species of a spectrum and the species predicted for this spectrum, for example, defined as being equal to distance Ω(yᵢ, k) according to relation (7). Unlike micro-accuracy, accuracies per species, and macro-accuracy, which consider prediction errors as being of equal significance, the taxonomy cost enables to quantify the severity of each prediction error.

The following algorithms have been analyzed and compared:
“SVM_one-vs-all”: algorithm according to relations (11), (12), (13), (14):
“SVM_cost_{—}0-1”: algorithm according to relations (20), (21), (22), (23);
“SVM_cost_taxo”: algorithm according to relations (20), (21), (22bis), and (23) with f (Δ(yᵢ,k),ξᵢ) defined according to relations (6) and (7);
“SVM_struct_{—}0-1”: algorithm according to relations (2), (3), (4), (8)-(10) with f(Δ(yᵢ,k),ξᵢ)=1−ξᵢ;:
“SVM_struct_taxo”: algorithm according to relations (2), (3), (4), (8)-(10) with f (Δ(yᵢ, k),ξᵢ) defined according to relations (6) and (7).

The parameter C retained for each of these algorithms is that providing the best micro-accuracy and macro-accuracy.

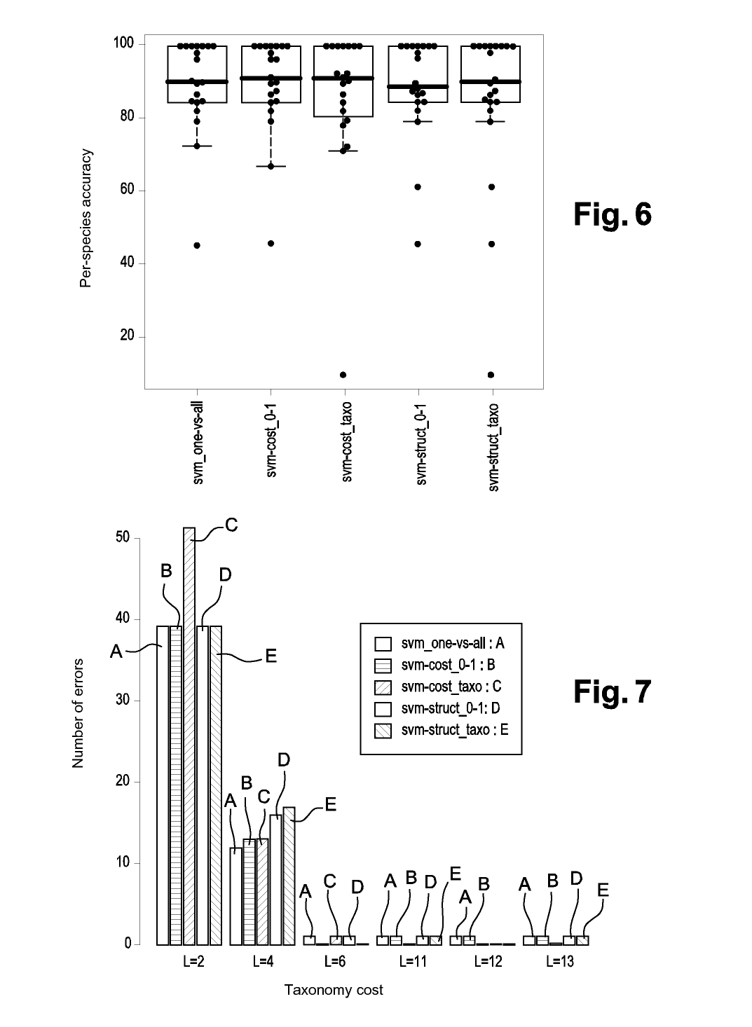

The following table lists for each of these algorithms the micro-accuracy and the macro-accuracy. FIG. 6 illustrates the accuracy per species of each of the algorithms, FIG. 7 illustrates the number of prediction errors according to the taxonomy cost thereof for each of the algorithms.

| SVM algorithm | Micro-accuracy | Macro-accuracy |
|---|---|---|
| SVM_one-vs-all | 90.4 | 89.2 |
| SVM_cost_0-1 | 90.4 | 89.0 |
| SVM_cost_taxo | 88.6 | 86.0 |
| SVM_struct_0-1 | 89.2 | 88.5 |
| SVM_struct_taxo | 90.4 | 89.2 |

These results, and particularly the above table and FIG. 6, show that both the representation of the data in accordance with the hierarchical representation and the loss functions have an incidence on the accuracy of the predictions, in terms of micro-accuracy as well as of macro-accuracy. It should be noted on this regard that the “SVM_struct_taxo” algorithm of the invention competes equally, for the least, with the conventional “one-versus-all” algorithm. However, as shown in FIG. 7, the prediction errors of the algorithms have different severities. Particularly, the “SVM_one-vs-all” and “SVM_cost_{—}0-1” algorithms, which take into account no hierarchical representation between reference species, generate prediction errors of high severity. The algorithm making the smallest number of severe errors is the “SVM_cost_taxo” algorithm, no taxonomy cost error greater than 4 having been detected. However, the “SVM_cost_taxo” algorithm has a lower performance in terms of micro-accuracy and of macro-accuracy.

It can thus be deduced from the foregoing that the introduction of a priori information in the form of a hierarchical representation, particularly a taxonomy and/or clinical phenotype representation, of the reference species and of quantitative distances between species in the form of loss functions enables to manage the tradeoff between, on the one hand, the global accuracy of the identification of unknown microorganisms and, on the other hand, the severity of identification errors.

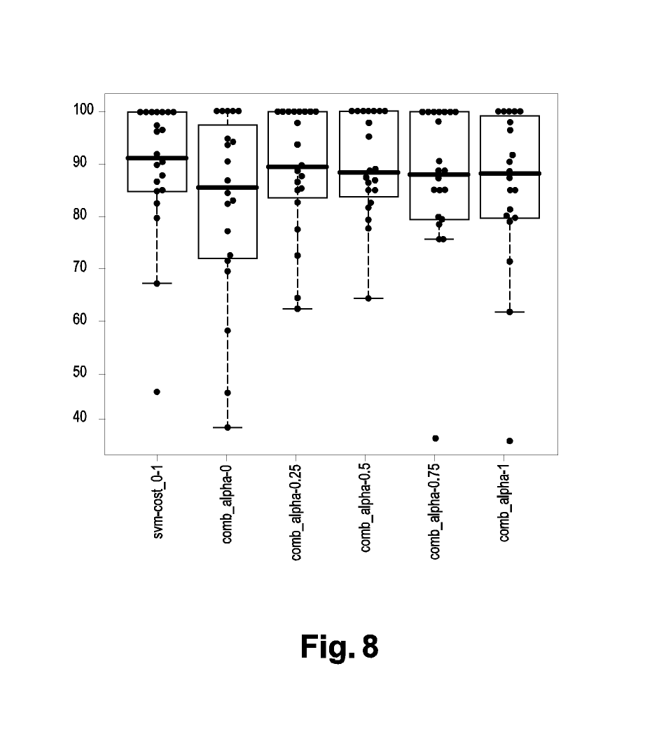

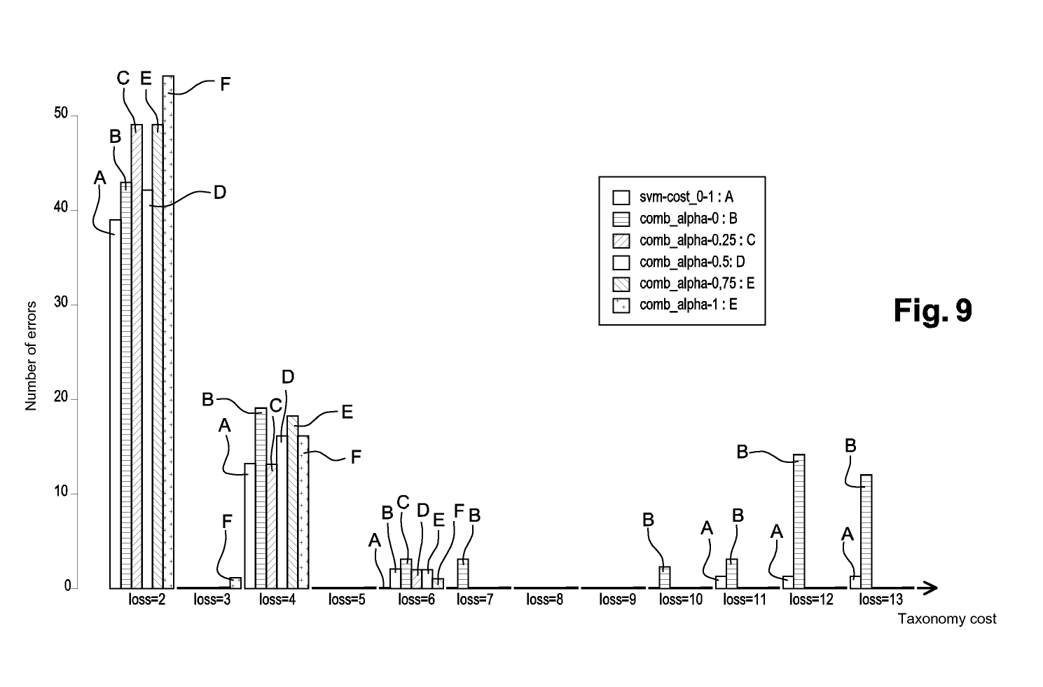

Analyses have also been made on loss functions equal to a convex combination of the distance in the tree and confusion loss function according to relation (19), more particularly for the “SVM_cost_taxo_conf” algorithm according to relations (20), (21), (22bis). Function f(Δ(yᵢ, k),ξᵢ) is defined according to relation (6) and loss functions Δ(yᵢ, k) are calculated by implementing the second example of the method of calculating loss functions Δ(yᵢ, k), with Δ(yᵢ, k) being defined according to relations (18) and (19), replacing the inter-node confusion matrix with the inter-species confusion matrix. The “SVM_cost_taxo_conf” algorithm has been implemented for different values of parameter a , that is, values 0, 0.25, 0.5, 0.75, and 1, parameter in relation (18) being equal to 1, and parameter C in relation (20) being equal to 1,000. The results of this analysis are illustrated in FIGS. 8 and 9, which respectively illustrate the accuracies per species and the taxonomy costs for the different values of parameter α. These drawings also illustrate, for comparison purposes, the accuracies per species and the taxonomy costs of the “SVM_cost_{—}0/1” algorithm.

As can be noted in the drawings, when parameter α comes close to one, the loss functions being thus substantially defined only by the distance in the tree of the hierarchical representation, the accuracy decreases and the severity of errors increases. Similarly, when parameter α comes close to zero, the loss functions being substantially defined from a confusion matrix only, the accuracy per species decreases and the severity of errors increases.

However, for values of parameter α within range [0.25; 0.75] , and particularly within range [0.25; 0.5] , a greater accuracy can be observed, the lowest accuracy per species being greater by 60% than the lowest accuracy per species of the SVM_cost_{—}0/1 algorithm. A substantial decrease of severe prediction errors, and particularly having a taxonomy cost greater than 6, can also be observed. Further, it can be observed that for values of a close to 0.5, particularly for value 0.5 illustrated in the drawings, the number of errors having a taxonomy cost equal to 2 is decreased as compared with the number of errors of same cost with values of a close to 0.25.

Preliminary analyses show a similar impact for a “SVM_struct_taxo_conf” algorithm implementing relations (2), (3), (4), (8)-(10) with, as a function f(Δ(yᵢ,k),ξᵢ), that defined at relation (6) and, as loss functions Δ(yᵢ,k) , those calculated by implementing the second example of the method of calculating loss functions Δ(yᵢ,k) by using relations (18) and (19).

Embodiments applied to MALDI-TOF-type mass spectrometry have been described. These embodiments apply to any type of spectrometry and spectroscopy, particularly, vibrational spectrometry and autofluorescence spectroscopy, only the generation of training vectors, particularly the pre-processing of spectra, being likely to vary.

Similarly, embodiments where the spectra used to generate the training data have no structure have been described.

Now, the spectra are “structured” by nature, that is, their components, the peaks, are not interchangeable. Particularly, a spectrum comprises an intrinsic sequencing, for example, according to the mass-to-charge ratio for mass spectrometry or according to the wavelength for vibrational spectrometry, and a molecule or an organic compound may give rise to a plurality of peaks.

According to the present invention, the intrinsic structure of the spectra is also taken into account by implementing non-linear SVM-type algorithms using symmetrical kernel functions K(x, y) defined as being positive, quantifying the structure similarity of a pair of spectra (x, y). Scalar products between two vectors appearing in the above-described SVM algorithms are then replaced with said kernel functions K(x, y). For more details, reference may for example be made to chapter 11 of document “Kernel Methods for Pattern Analysis” by John Shawe-Taylor & Nello Cristianini—Cambridge University Press, 2004.

1. A method of identifying by spectrometry unknown microorganisms from among a set of reference species, comprising:
a first phase of supervised learning of a reference species classification model, comprising:
for each species, acquiring a set of training spectra of identified microorganisms belonging to said species;
transforming each acquired training spectrum into a set of training data according to a predetermined format for their use by a multi-class support vector machine type algorithm; and
determining the classification model of the reference species as a function of the sets of training data by means of said algorithm of multi-class support vector machine type,
a second step of predicting an unknown microorganism to be identified, comprising:
acquiring a spectrum of the unknown microorganism; and
applying a prediction model according to said spectrum and to the classification model to infer at least one type of microorganism to which the unknown microorganism belongs,
characterized in that:
the transforming of each acquired training spectrum comprises:
transforming the spectrum into a data vector representative of a structure of the training spectrum;
generating the set of data according to the predetermined format by calculating the tensor product of the data vector by a predetermined vector bijectively representing the position of the reference species of the microorganism in a tree-like hierarchical representation of the reference species in terms of evolution and/or of clinical phenotype;
and the classification model is a classification model of classes corresponding to nodes of the tree of the hierarchical representation, the algorithm of multi-class support vector machine type comprising determining parameters of the classification model by solving a single problem of optimization of a criterion expressed according to the parameters of the classification model under margin constraints comprising so-called “loss functions” quantifying a proximity between the tree nodes.

2. The identification method of claim 1, characterized in that loss functions associated with pairs of nodes are equal to distances separating the nodes in the tree of the hierarchical representation.

3. The identification method of claim characterized in that loss functions associated with pairs of nodes are respectively greater than distances separating the nodes in the tree of the hierarchical representation.

4. The identification method of claim 1, characterized in that the loss functions are calculated:
by setting the loss functions to initial values;
by implementing at least one iteration of a process comprising:
executing an algorithm of multi-class support vector machine type to calculate a classification model according to current values of the loss functions;
applying a prediction model according to the calculated classification model and to a set of calibration spectra of identified microorganisms belonging to the reference species, different from the set of training spectra;
calculating a classification performance criterion for each species according to results returned by said application of the prediction model to the set of calibration spectra; and
calculating new current values of the loss functions by modifying the current values of the loss functions according to the calculated performance criteria.

5. The identification method of claim 4, characterized in that:
the calculation of the performance criterion comprises calculating a confusion matrix as a function of the results returned by said application of the prediction model;
and the new current values of the loss functions are calculated as a function of the confusion matrix.

6. The identification method of claim 4, characterized in that:
the calculation of the performance criterion comprises calculating a confusion matrix as a function of the results returned by said application of the prediction model;
and the new current values of the loss functions respectively correspond to the components of a combination of a first loss matrix listing distances separating the reference species in the tree of the hierarchical representation and of a second matrix calculated as a function of the confusion matrix.

7. The identification method of claim 6, characterized in that the current values of the loss functions are calculated according to relation:
Δ(yᵢ, k)=α×Ω(yᵢ, k)+(1−α)×Δ_{confusion} (yᵢ, k)
where Δ(yᵢ, k) are said current values of the loss functions for pairs of nodes (yᵢ, k) of the tree, Ψ(yᵢ, k) and Δ_{confustion} (yᵢ, k) respectively are the first and second matrixes, and α is a scalar between 0 and 1.

8. The identification method of claim 7, characterized in that scalar α is between 0.25 and 0.75.

9. The identification method of claim 4, characterized in that the initial values of the loss functions are set to zero for pairs of different nodes and equal to 1 otherwise.

10. The identification method of claim 1, characterized in that a distance Ψ separating two nodes n₁, n₂ in the tree of the hierarchical representation is determined according to relation:
Ψ(n₁, n₂)=depth(n₁)+depth(n₂)−2×depth(LCA(n₁, n₂))
where depth(n₁) and depth(n₂) respectively are the depth of nodes n₁, n₂, and depth(LCA(n₁, n₂)) is the depth of the closest common ancestor LCA(n₁, n₂) of nodes n₁, n₂ in said tree.

11. The identification method of claim 1, characterized in that the prediction model is a prediction model for the nodes of the trees to which the unknown microorganism to be identified belongs.

12. The identification method of claim 1, characterized in that the optimization problem is formulated according to relations:
$${min}_{W , {ξ}_{i}} \frac{1}{2} {‖ W ‖}^{2} + C {\sum }_{i = 1}^{N} {ξ}_{i}$$
under constraints:
ξᵢ≧0, ∀i ∈ [1, N] [symbol]
W, Ψ(xᵢ, yᵢ [symbol] ≧ [symbol] W, Ψ(xᵢ, k) [symbol] +f (Δ(yᵢ, k),ξᵢ), ∀i ∈ [1, N], ∀k ∈ Y\yᵢ
in which expressions:
N is the number of training spectra;
K is the number of reference species;
T is the number of nodes in the tree of the hierarchical representation and Y=[1, T] is a set of integers used as reference numerals for the nodes of the tree of the hierarchical representation;
W ∈ [symbol] ^{p×T} is the concatenation (w₁w₂ . . . w_{T}^{T} of weight vectors w₁, w₂, . . . , w_{T} ∈ [symbol] ^{p} respectively associated with the nodes of said tree, p being the cardinality of the vectors representative of the structure of the training spectra;
C is a scalar having a predetermined setting;
∀i ∈ [1, N] is a scalar;
X={xᵢ}, i ∈ [1, N] is a set of vectors xᵢ ∈ [symbol] ^{p} representative of the training spectra;
∀i ∈ [1, N] yᵢ is the reference of the node in the tree of the hierarchical representation corresponding to the reference species of training vector xᵢ ;
Ψ(x,k)=x [symbol] Λ(k) where:
x ∈ [symbol] ^{p} is a vector representative of a training spectrum;
Λ(k) ∈ [symbol] ^{T} is a predetermined vector bijectively representing the position of reference node k ∈ Y in the tree of the hierarchical representation; and [symbol]
: [symbol] ^{p}× [symbol] ^{T}→ [symbol] ^{p×T} is the tensor product between space [symbol] ^{p} and space [symbol] ^{T}; [symbol]
W, Ψ [symbol] is the scalar product over space Σ^{p×T};
Δ(yᵢ, k) is the loss function associated with the pair of nodes bearing respective references yᵢ and k in the tree of the hierarchical representation;
f (Δ(yᵢ, k),ξᵢ) is a predetermined function of scalar ξᵢ and of loss function Δ(yᵢ, k); and
symbol “\” designates exclusion.

13. The identification method of claim 12, characterized in that function f (Δ(yᵢ, k),ξᵢ) is defined according to relation:
f(Δ(yᵢ, k),ξᵢ)=Δ(yᵢ, k)−ξᵢ

14. The identification method of claim 12, characterized in that function f (Δ(yᵢ, k),ξᵢ) is defined according to relation:
$$f ( Δ ( {y}_{i} , k ) , {ξ}_{i} ) = 1 - \frac{{ξ}_{i}}{Δ ( {y}_{i} , k )}$$

15. The identification method of claim 12, characterized in that the prediction step comprises:
transforming the spectrum of the unknown microorganism to be identified into a vector xₘ, according to the predetermined format of the algorithm of multi-class support vector machine type;
applying a prediction model according to relations:
T_{indent}=arg maxₖ (s(xₘ, k)) k ∈ [1, T]
where T_{indent} is the reference numeral of the node of the hierarchical representation identified for the unknown microorganism,
s(xₘ, k)= [symbol] W, Ψ(xₘ, k) [symbol] and Ψ(xₘ, k)=xₘ [symbol] Λ(k).

16. A device for identifying a microorganism by mass spectrometry, comprising:
a spectrometer capable of generating mass spectra of microorganisms to be identified;
a calculation unit capable of identifying the microorganisms associated with the spectra generated by the spectrometer by implementing the prediction step of claim 1.

17. The identification method of claim 7, characterized in that scalar a is between 0.25 and 0.5.
